(19) United States
(12) Patent Application Publication
(10) Pub. No.: US 2026/0053970 A1
(43) Pub. Date: Feb. 26, 2026

(54) AIR STERILISATION UNIT AND METHOD FOR STERILISING AIR

(71) Applicant: EIRCHEMICALS LTD, Magherafelt, County Derry (GB)

(72) Inventor: Martin Owen KEARNEY, Magherafelt, County Derry (GB)

(21) Appl. No.: 18/865,594

(22) PCT Filed: May 11, 2023
(86) PCT No.: PCT/EP2023/062684

§ 371 Date: Nov. 13, 2024

(30) Foreign Application Priority Data
May 13, 2022 (GB) 2207006.4

(51) Int. Cl. A61L 9/14; A61L 2/10; A61L 2/18; A61L 9/20; A61L 101/02; B01D 46/00

(52) U.S. Cl. CPC A61L 9/145; A61L 2/10; A61L 2/186; A61L 9/20; B01D 46/0028; B01D 46/0047; A61L 2101/02; A61L 2202/11; A61L 2209/12; A61L 2209/134; A61L 2209/14; A61L 2209/16; B01D 2273/30; B01D 2279/35; B01D 2279/50; B01D 2279/65

(57) ABSTRACT

An air sterilisation unit comprising: a housing having an air intake arranged to allow contaminated air to enter the housing, and an air outlet arranged to allow sterilised air to exit the housing; and air sterilisation assembly arranged to sterilise air within the housing by entrapping or removing particulate matter from the air flowing from the air intake to the air outlet.

The present invention relates to an air sterilisation unit.

Studies have shown that micro-organisms, such as bacteria, viruses and fungi, are not killed effectively using standard air cleaning techniques. For example, air filtration systems which use filters to remove particulate matter from air capture such micro-organisms in the filter material. Often the captured micro-organisms can live in the filter material where they will multiply and potentially be released back into the air once the filter material becomes saturated. Standard air filtration techniques catch rather than kill micro-organisms, and do not fully address the problem of permanent removal of harmful micro-organisms from an environment.

Hydrogen peroxide technology can be used to provide more effective cleaning than standard air filtration techniques. Hydrogen peroxide is an oxidising agent which oxidises the cells or spores of micro-organisms on contact, thus deactivating them. Hydrogen peroxide sterilisation via vapour technology has a proven efficacy rating of 99.99%. Decontaminating air with Hydrogen Peroxide Vapour (HPV) technology (referred to as “fogging”) has become widely accepted in public institutions and is beneficial in lowering health-care-associated infections.

Existing fogging technology consists of a mobile vapour generator which mists aqueous hydrogen peroxide to facilitate disinfection of rooms, areas and equipment. A very fine layer of micro-condensation is formed on exposed surfaces, deactivating any micro-organisms present. A significant drawback of using hydrogen peroxide in this way is that the vapour is extremely reactive and a residual rate of hydrogen peroxide over 10 mg/m³ is hazardous to human health. As such, during the cleaning process the room or area to be cleaned must be completely evacuated and remain unused for up to 24 hours. Furthermore, existing fogging systems typically consume large amounts of hydrogen peroxide. Having to repeatedly transport large quantities of hydrogen peroxide to a room or area to be cleaned has a negative environmental impact.

It is an object of the present invention to obviate or mitigate the problems outlined above. In particular, it is an object of the present invention to provide a sterilisation system which can be easily deployed.

It is a further object of the invention to provide a method of sterilising air that does not require complete evacuation of a room or space.

It is a further object of the invention to provide a portable solution for air sterilisation.

It is a further object of the invention to provide a means for removing harmful micro-organisms from the air present in a room, area or locality.

It is a further object of the invention to provide an environmentally friendly air sterilisation unit.

According to a first aspect of the invention there is provided an air sterilisation unit comprising: a housing; an air intake means for allowing air to enter the housing; an air outlet means for allowing air to exit the housing; and a means for sterilising air within the housing.

Advantageously, the air sterilisation unit allows contaminated air to enter the housing, is able to effectively deactivate airborne micro-organisms present in the air inside the housing and allows clean air to leave the housing. Further advantageously, sterilisation of the air is confined to the air sterilisation unit. Ideally, the air sterilisation unit provides contained hydrogen peroxide fogging.

Preferably, the means for sterilising air within the housing is configured or configurable to entrap and/or remove particulate matter and/or carbon dioxide from air within the housing.

Advantageously, this prevents contaminated air from being dumped back into the environment. This is in comparison to air filtration units or HVAC systems which rely on filters to remove particulate matter from the air.

By particulate matter we mean airborne micro-organisms, viruses, bacteria, dust, pathogens, VOCs and/or any other undesirable matter suspended in the airflow.

Preferably, the air sterilisation unit is adapted to remove carbon dioxide from air within the housing. Ideally, the air sterilisation unit removes carbon dioxide from air via water absorption.

Preferably, the air sterilisation unit comprises an air sterilisation chamber.

Preferably, the air sterilisation unit or the means for sterilising air within the housing comprises a fluid supply means for supplying fluid within the air sterilisation chamber.

Preferably, the fluid supply means is configurable to generate a flow of fluid within the housing.

Preferably, the flow of fluid is counter current to the flow of air within the housing.

Preferably, the fluid supply means is configurable to supply fluid to contact the air within the housing.

Ideally, the means for sterilising air within the housing is configurable to remove particulate matter from the air within the housing via the induction of counter current flow between a fluid and the air within the housing.

Preferably, the fluid supply means is configurable to supply droplets of fluid to air within the housing.

Preferably, the fluid is a solvent configurable to absorb and/or entrap particulate matter suspended in the air within the housing.

Preferably, the fluid is a liquid.

Ideally, the fluid comprises water. Ideally, the fluid comprises less than 9% hydrogen peroxide. Ideally, the fluid comprises less than 8%, less than 7%, less than 6%, less than 5%, less than 4% or less than 3% hydrogen peroxide.

Ideally, the fluid and/or droplets are configured to absorb and/or entrap particulate matter in the air within the housing.

Advantageously, this causes separation of the particulate matter from the air.

Preferably, the fluid and/or droplets are configured to remove particulate matter from the air within the housing.

Preferably, the air sterilisation unit comprises a fluid delivery means.

Preferably, the fluid delivery means is configured to supply fluid from one or more parts of the air sterilisation unit to one or more other parts of the air sterilisation unit including the fluid supply means.

Ideally, the fluid delivery means comprises one or more pumps and/or conduits and/or any other means configurable to deliver fluid.

Preferably, the means for sterilising air within the housing comprises a means for manipulating fluid-particle contact within the housing.

Preferably, the means for sterilising air within the housing comprises means for increasing fluid-particle contact within the housing.

By fluid-particle contact we mean the occurrence and/or duration of contact between the fluid and particulate matter suspended in the air within the housing and/or between the fluid and the air within the housing.

Ideally, the means for manipulating fluid-particle contact within the housing is configurable to increase fluid-particle contact within the housing.

Preferably, the means for manipulating fluid-particle contact within the housing is configurable to manipulate the speed and/or direction of airflow through the housing.

Ideally, the means for manipulating fluid-particle contact within the housing is configurable to reduce the speed of airflow through the housing.

Advantageously, this increases fluid-particle and/or solvent-particle contact. Thereby, this increases the likelihood of separation of particulate matter and/or carbon dioxide from the air within the housing. Further advantageously, this means that air and particulate matter and/or carbon dioxide within the housing will be exposed to fluid for a longer period of time. This is in comparison to not having a means for manipulating fluid-particle contact within the housing.

Preferably, the means for sterilising air within the housing comprises a scrubbing tower.

Preferably, the air sterilisation unit is configurable to perform scrubbing of air to sterilise the air and/or to remove particulate matter and/or carbon dioxide from the air.

Preferably, the air sterilisation unit comprises a fluid scrubbing tower.

Ideally, the air sterilisation unit is configurable to perform fluid scrubbing of air to provide air sterilisation.

Advantageously, this provides an environmental solution to air sterilisation via a scrubbing tower.

Preferably, the air sterilisation unit comprises packing material.

Preferably, the means for manipulating fluid-particle contact within the housing and/or the scrubbing tower comprises packing material.

Ideally, the packing material comprises distillation column packing filling springs, porous ceramic material and/or fibrous media, or any other suitable material or combinations thereof.

The packing material may be coated or uncoated and may be reactive to UVC lamps to enhance sterilisation of air.

Preferably, the packing material is configurable to manipulate the speed and/or direction of airflow through the housing.

Ideally, the packing material is configurable to slow airflow through the housing such that fluid-particle contact and/or solvent-particle contact is increased.

Advantageously, the packing material can be configured to slow airflow through the housing such that fluid-particle contact and/or solvent-particle contact is increased.

Preferably, the packing material is adapted to provide an elongated path for air flow and/or fluid flow.

Advantageously, having an elongated path means that the air and/or fluid within the housing takes longer to pass through the housing, and therefore the air will be exposed to the fluid for an increased amount of time. Thereby, this increases contact between the air and fluid to ensure adequate removal of particulate matter and/or carbon dioxide from the air.

Preferably, the air sterilisation unit comprises a fluid reservoir configured to contain fluid.

Preferably, the air sterilisation unit comprises a fluid flow. Throughout the specification the terms fluid flow, flow of fluid and fluid flow path are used interchangeably.

Preferably, the fluid delivery means is configurable to deliver droplets of fluid to the means for sterilising air within the housing.

Preferably, the fluid flow is configurable to carry particulate matter to the fluid reservoir.

Ideally, the fluid and/or droplets are configurable to carry particulate matter to the fluid reservoir.

Preferably, the fluid flow is configurable to carry particulate matter via gravity.

Ideally, the fluid and/or droplets are configurable to carry particulate matter to the fluid reservoir via gravity.

Preferably, the air sterilisation unit comprises a fluid sterilisation means.

Ideally, the fluid reservoir comprises a fluid sterilisation means.

Preferably, the fluid sterilisation means is configurable to sterilise and/or disinfect fluid within the fluid reservoir.

Advantageously, fluid containing particulate matter can be sterilised such that the particulate matter is deactivated and the fluid is suitable for re-use.

Preferably, the fluid sterilisation means comprises one or more UV lamps configurable to sterilise and/or disinfect the fluid.

Advantageously, after sterilisation and/or disinfection, the fluid can be fed back into the air sterilisation means for re-use. Therefore, this reduces the amount of fluid being consumed by the air sterilisation unit. Further, this reduces costs and operator involvement in relation to the management of the air sterilisation unit.

Additionally, or alternatively, the fluid sterilisation means comprises a sterilisation agent.

Preferably, the fluid sterilisation means comprises a means for supplying sterilisation agent.

Advantageously, the system having its own means for generating sterilisation agent means that the amount of an external source of sterilisation agent that is required is reduced.

This reduces the cost and hazards that would arise from transporting large amounts of sterilisation agent to the unit.

Preferably the means for supplying a sterilisation agent is located within the housing.

Preferably the means for supplying a sterilisation agent is located within the fluid reservoir.

Preferably the means for generating sterilisation agent is adapted for continuous production of sterilisation agent.

Preferably, the fluid sterilisation means comprises multiple means for generating one or more sterilisation agents.

Preferably, the means for generating sterilisation agent is adapted for continuous production of hydrogen peroxide and/or singlet oxygen.

Preferably the means for generating sterilisation agent continuously produces sterilisation agent, in use.

Ideally, the fluid sterilisation means comprises a hydrogen peroxide reactor.

Preferably, the hydrogen peroxide reactor is configurable to produce hydrogen peroxide from one or more base materials such as hydrogen, oxygen and water.

Ideally, the concentration of hydrogen peroxide in the fluid is less than 9%. The fluid may comprise hydrogen peroxide that has been added to the air sterilisation unit and/or hydrogen peroxide that has been generated by the air sterilisation unit, most preferably, by the hydrogen peroxide reactor.

Preferably, the sterilisation agent is hydrogen peroxide.

Additionally, or alternatively again, the sterilisation agent is or comprises singlet oxygen.

Optionally, the fluid sterilisation means comprises a singlet oxygen generator.

Preferably, the fluid sterilisation means is adapted to sterilise fluid in the reservoir via production of singlet oxygen.

Ideally, the fluid sterilisation means comprises one or more base materials and/or reactants configurable to react to produce singlet oxygen.

Preferably, the fluid sterilisation means comprises activation means configurable to activate the one or more base materials.

Preferably, the fluid sterilisation means is configurable to initiate photochemical, thermal, chemical, or enzymatic activation of oxygen or dioxygen to produce singlet oxygen.

Preferably, the one or more base materials are one or more photosensitisers.

Ideally, the activation means comprises a light source.

Ideally, the activation means comprises one or more LEDs and/or UV lamps.

Preferably, the fluid sterilisation means comprises a base material reservoir.

Advantageously, the base material reservoir is able to store a ready supply of the base material(s) used to make the sterilisation agent.

Preferably, the fluid sterilisation means comprises a sterilisation agent reservoir. Advantageously, the sterilisation agent reservoir is able to store a ready supply of the sterilisation agent for use within the fluid sterilisation means and/or fluid reservoir.

Preferably, the sterilisation agent is locatable within the sterilisation agent reservoir.

Preferably, the means for supplying a sterilisation agent is adapted to output sterilisation agent to the sterilisation agent reservoir, such as via a tube or conduit.

Ideally, the sterilisation agent is configurable to sterilise and/or disinfect fluid within the fluid reservoir.

Advantageously, sterilisation is maintained within the housing.

Preferably, the sterilisation agent is configurable to deactivate particulate matter suspended in the fluid.

Preferably, the air sterilisation unit comprises one or more heaters configurable to heat the fluid and/or water within the fluid reservoir.

Preferably, the air sterilisation unit comprises a further means for sterilising air within the housing.

Ideally, the further means for sterilising air within the housing comprises one or more UV lamps adapted to sterilise air within the housing.

Preferably, the air sterilisation unit is portable.

Preferably, the air sterilisation unit is a tower.

Preferably, the air sterilisation unit is a standalone unit.

Optionally, the air sterilisation unit forms part of an air conditioning system.

Preferably, the housing comprises one or more side walls.

Preferably, the housing comprises a lower member or base.

Preferably, the housing comprises an upper member or lid.

Preferably, the or each side wall extends between the lower member or base and the upper member or lid.

Preferably, the air sterilisation unit comprises support means. Advantageously, the support means is adapted to hold the sterilisation unit, and particularly the air intake means, at an appropriate height off the ground.

Preferably, the support means is adapted to support the housing.

Preferably, the support means comprises a support base and/or one or more support legs.

Preferably, the support means is locatable at or adjacent to the lower member of the housing.

Ideally, at least part or all of the means for sterilising air within the housing is located within the air sterilisation chamber.

Preferably, the air intake means comprises one or more intake apertures.

Preferably, the or each intake aperture is adapted to allow air to pass into the housing.

Preferably, air is able to enter the means for sterilising air within the housing via the air intake means.

Preferably, the housing comprises an air intake chamber.

Ideally, the means for sterilising air within the housing comprises an air intake chamber.

Preferably, air is able to enter the air intake chamber via the air intake means.

Preferably, the air intake means comprises one or more intake apertures adapted to allow air to pass into the intake chamber.

Preferably, the air intake means comprises a plurality of intake apertures.

Preferably, the air intake means is located in a side wall of the housing.

Preferably, the air intake means is located in a middle or lower portion of a housing side wall.

Preferably, the means for sterilising air within the housing is located in a middle or lower portion of the housing.

Preferably, the air intake means is located in a portion of a housing side wall adjacent to the air intake chamber.

Preferably, a plurality of intake apertures surrounds a portion of the housing side wall. Advantageously, having a plurality of intake apertures allows the air sterilisation unit, and particularly the air intake means, to draw air into the air sterilisation unit from a plurality of directions.

### Preferably, the Air Intake Means Is Configured to Allow Air to Pass Into the Housing From a plurality of directions.

Preferably the air outlet means comprises one or more outlet apertures.

Preferably the or each outlet aperture is adapted to allow air to pass out of the housing.

Preferably, the air sterilisation unit comprises an air outlet chamber.

Preferably the air outlet means is located in the upper member or lid of the housing.

Ideally, the air outlet means is located adjacent to the air outlet chamber.

Ideally, the means for sterilising air within the housing is located between the air intake means and the air outlet means.

Preferably, the air intake means is located adjacent to a lowermost portion of the air sterilisation chamber.

Preferably, the air sterilisation unit comprises an air flow path.

Preferably, the air intake means is in fluid communication with the air outlet means via the air flow path.

Preferably, air is able to flow through the housing via the air flow path.

Preferably, the air flow path is located entirely or at least partially within the housing.

Preferably, the air flow path is located at least partially within the means for sterilising air within the housing.

Preferably, the air flow path is located at least partially within the air sterilisation chamber.

Advantageously, this ensures that air flows through the air sterilisation chamber and/or means for sterilising air within the housing so that it can be sterilised.

Preferably, the air sterilisation unit comprises a droplet separator.

Ideally, the droplet separator is adapted to separate any fluid entrained in the air within the housing.

Advantageously, this prevents the escape of any particulate matter and/or hydrogen peroxide that has been captured by the fluid from escaping the air sterilisation unit via fluid entrained in the air.

Preferably, the droplet separator is located between the air sterilisation chamber and the air outlet chamber.

Preferably, the further means for sterilising air within the housing is located within the air outlet chamber.

Preferably, the air sterilisation unit comprises a dehumidifier.

Advantageously, any unwanted moisture or fluid will be removed by the dehumidifier.

Further advantageously, this prevents particulate matter and/or hydrogen peroxide that has been captured by the fluid from escaping the air sterilisation unit.

Ideally, the dehumidifier is located above the fluid supply means.

Advantageously, fluid from the fluid supply means, intended to flow through the air sterilisation chamber, will not be immediately removed by the dehumidifier.

Preferably, the dehumidifier is located above the air sterilisation chamber and/or means for sterilising air within the housing.

Preferably, the dehumidifier is located within the air outlet chamber.

Advantageously, any excess moisture in the air flowing through the air outlet chamber will be removed prior to the air exiting the air sterilisation unit via the air outlet means.

Ideally, the dehumidifier is located above the droplet separator.

Advantageously, any fluid or moisture which manages to bypass the droplet separator will be exposed to the dehumidifier.

Preferably, air is able to enter the air sterilisation unit via the air intake means, travel through the air sterilisation chamber, through the droplet separator, through the air outlet chamber and out of the air outlet means.

Preferably, the air sterilisation unit comprises a fluid flow path.

Preferably, fluid is able to flow through the housing via the fluid flow path.

Preferably, the fluid flow path is located entirely or at least partially within the housing.

Preferably, the fluid flow path is located at least partially within the means for sterilising air within the housing.

Advantageously, this ensures exposure of the air flow path to the fluid flow path. Further, this ensures contact between the fluid in the fluid flow path and air and/or particulate matter or carbon dioxide in the air flow path.

Ideally, the fluid flow path runs countercurrent to the air flow path.

Preferably, the fluid supply means is in fluid communication with the fluid reservoir via the fluid flow path.

Preferably, the fluid supply means comprises one or more fluid outlet apertures.

Preferably, the one or more fluid outlet apertures are located at an uppermost portion of the air sterilisation chamber and/or of the scrubbing tower.

Ideally, the fluid supply means is located above the means for manipulating fluid-particle contact within the housing and/or above the packing material.

Preferably, the fluid reservoir is located below the air sterilisation chamber and/or the scrubbing tower.

Ideally, the fluid reservoir is located below the means for manipulating fluid-particle contact within the housing and/or the packing material.

Advantageously, fluid from the fluid supply means can flow from the fluid outlet aperture(s) through the housing, air sterilisation chamber and/or means for sterilising air within the housing via gravity to the fluid reservoir.

Preferably, the fluid reservoir is located below the air intake means and/or air intake chamber.

Advantageously, fluid flows through the air intake chamber to begin sterilising air as soon as the air enters the housing.

Preferably, the fluid delivery means is configurable to deliver sterilised fluid from the fluid reservoir to the fluid supply means and/or one or more fluid outlet apertures.

Preferably, the means for manipulating fluid-particle contact within the housing increases the length and/or duration of the air flow and/or fluid flow path.

Advantageously, this means that air within the housing is exposed to the fluid for a longer period of time. Thereby, this increases absorption and/or entrapment of particulate matter or carbon dioxide by the fluid. Further, as hydrogen peroxide coats the packing material, the longer exposure means more hydrogen peroxide will evaporate off the packing material when contacted by the air, thereby improving the efficacy of the air sterilisation unit.

Preferably the air sterilisation unit comprises air flow generation means.

Advantageously, air flow generation means allows air to be drawn through the air sterilisation unit.

Preferably, the air flow generation means is adapted to generate a fluid flow within, through and/or along the air flow path.

Preferably, the air flow generation means is adapted to generate a fluid flow in a direction from the air intake means towards the air outlet means.

Preferably, the air flow generation means is adapted to cause air to flow through the air sterilisation unit.

Preferably, the air flow generation means is adapted to cause air to flow into the air sterilisation unit via the air intake means.

Preferably, the air flow generation means is configured to allow air to flow through the air flow generation means.

Preferably, the air flow generation means is adapted to cause air to flow into and/or through the housing and/or means for sterilising air within the housing. Advantageously, un-sterilised air can be drawn into the air sterilisation unit by the air flow generation means so that said air can be sterilised.

Preferably, the air flow generation means is located within the housing.

Preferably, the air flow generation means is adapted to cause air to flow into, through and/or out of the air outlet chamber.

Preferably, the air flow generation means is adapted to cause air to flow out of the air sterilisation unit via the air outlet means.

Preferably, the air flow generation means is adapted to cause air to flow out of the housing and/or air sterilisation chamber.

Preferably, the air flow generation means is located within or adjacent to the air outlet chamber.

Preferably, the air flow generation means is adjacent to the air outlet means.

Preferably, the air sterilisation unit comprises a plurality of air flow generation means.

Preferably, the or each air flow generation means comprises a fan.

Preferably, the or each air flow generation means comprises a motor.

Preferably, the air sterilisation unit comprises a filtration means.

Preferably, the air sterilisation unit comprises a fluid filtration means. Advantageously, fluid filtration means can be used to remove particulate matter and/or sediment from fluid travelling into, through, within and/or out of the fluid reservoir.

Preferably the fluid filtration means is removable and/or replaceable. Advantageously, used and/or clogged fluid filtration means can be replaced with new and pristine fluid filtration means, providing better filtration.

Preferably, the air sterilisation unit comprises air filtration means.

Advantageously, air filtration means can be used to remove fine particles such as dust and pollen travelling into, through, within and/or out of the air sterilisation unit.

Preferably, the air sterilisation unit comprises an outer air filtration means.

Preferably the outer air filtration means is adapted to filter particles such as dust and/or pollen at the point of entry into the air sterilisation unit.

Preferably the outer air filtration means comprises a cover means for the air sterilisation unit.

Preferably the cover means is a fabric or synthetic material cover means.

Preferably the cover means comprises indicia.

Preferably the air sterilisation unit comprises one or more further air filtration means.

Preferably the air sterilisation unit comprises an intake air filtration means.

Preferably the intake air filtration means is locatable adjacent to the air intake means.

Preferably, the intake air filtration means is adapted to filter particles such as dust and/or pollen within the air sterilisation unit.

Preferably, the intake air filtration means is adapted to filter particles such as dust and/or pollen moving from the intake chamber towards the air outlet means.

Preferably, the intake air filtration means comprises a filter.

Preferably, the intake air filtration means comprises a filter locatable adjacent to the air intake means.

Ideally, the air sterilisation unit comprises a UVC lamp located at or about the air outlet.

Advantageously, this removes hydrogen peroxide from the outlet air.

Preferably, the air filtration means comprises at least one filter.

Preferably, the air filtration means comprises a HEPA filter.

Preferably, water and sterilisation agent within the unit is recycled.

Optionally, the air sterilisation unit comprises one or more sensors. Advantageously, sensors can be used to measure one or more operating conditions of the unit.

Preferably, the air sterilisation unit comprises one or more sensors for detecting sterilisation agent outside the air sterilisation unit.

Preferably, the air sterilisation unit comprises one or more sensors for detecting sterilisation agent outside the housing.

Preferably, the air sterilisation unit comprises one or more sensors for detecting any escape of sterilisation agent from the air sterilisation unit.

Preferably, the air sterilisation unit comprises one or more sterilisation agent sensors adjacent to the outlet means.

Preferably, the air sterilisation unit comprises one or more sterilisation agent sensors on the outside of the housing.

Preferably, the air sterilisation unit comprises communication means such as wireless communication means.

Preferably, the communication means is connectable to a central hub or server.

Preferably, the communication means is adapted to send sensor data and/or performance information e.g. to a central hub or server.

Preferably, the air sterilisation unit is adapted to send and/or receive notifications.

Preferably, the air sterilisation unit is adapted to send a notification to a central hub or server when sterilisation agent is detected outside the housing.

Preferably, the air sterilisation unit comprises an inner chamber.

Preferably, the air sterilisation unit comprises an outer chamber.

Ideally, the outer chamber is configurable to house one or more controls.

Preferably, at least part of the fluid delivery means is located within the outer chamber.

Preferably, the air sterilisation unit is a closed loop air sterilisation unit. By ‘closed-loop’, we mean that sterilisation of the air is maintained within the air sterilisation unit.

Advantageously, this prevents escape of fluid, particulate matter, carbon dioxide and/or sterilisation agent from the air sterilisation unit.

Preferably, the air sterilisation unit is adapted to perform cyclic sterilisation of air.

Preferably, the air sterilisation unit is configurable to provide up to 1000 ft² with sterilised and/or conditioned air approximately or at least eight times per hour.

Preferably, the air sterilisation unit forms part of a heat recovery system and can function to sterilise air in a heat recovery system.

Ideally, the air sterilisation unit can operate with one or more other air sterilisation units.

Advantageously, multiple air sterilisation units can be grouped to and/or added to large HVAC systems to replace heat recovery units, recirculating air in a building by returning sterilised and/or conditioned air via a closed system. Further advantageously, multiple air sterilisation units can be grouped to regularly or irregularly to provide sterilised and/or conditioned air to areas larger than 1000 ft².

In an embodiment, the means for sterilising air within the housing is configured to activate hydrogen peroxide by using UV lamps to kill pathogens in the air located within the air sterilisation unit.

Ideally in this embodiment, the means for sterilising air comprises one or more UV lamps located in the air outlet chamber.

Ideally, the one or more UV lamps are located between the air flow generation means and the droplet separator and/or the fluid supply means.

Advantageously, the one or more UV lamps reduce hydrogen peroxide via activation and can reduce the level of hydrogen peroxide to less than 1 PPM.

Ideally in this embodiment, the air sterilisation unit comprises one or more reservoirs of hydrogen peroxide to supply hydrogen peroxide to the air within the air sterilisation unit. The reservoirs of hydrogen peroxide may contain a solution containing hydrogen peroxide and not necessarily pure hydrogen peroxide.

Ideally, the hydrogen peroxide is supplied to the air outlet chamber. Ideally, the reservoirs are configured to be manually topped up with hydrogen peroxide when needed.

Ideally, the air sterilisation unit comprises a means for supplying the hydrogen peroxide from the hydrogen peroxide reservoir to a space that contains UV light, most preferably, UV light from the one or more UV lamps.

### Ideally, the Means for Supplying the Hydrogen Peroxide Can Supply Hydrogen Peroxide to the air outlet chamber.

Ideally, the means for supplying the hydrogen peroxide can supply hydrogen peroxide a space located between the air flow generation means and the droplet separator and/or the fluid supply means.

Ideally, the means for supplying hydrogen peroxide is or comprises a wick.

According to a further aspect of the invention there is provided a replaceable cartridge for supplying a means for sterilising air within an air sterilisation unit.

Ideally, the means for sterilising air is a hydrogen peroxide water mix.

Ideally, the replaceable cartridge has a means for delivering the means for sterilising air into the air sterilisation unit.

Ideally, the replaceable cartridge comprises a wick.

Ideally, the means for sterilising air comprises a fluid supply means for supplying fluid into the air sterilisation chamber.

Preferably, the fluid supply means is configurable to generate a flow of fluid into the housing.

Preferably, the flow of fluid would be counter current to the flow of air within the housing.

Preferably, the fluid supply means is configurable to supply fluid to contact the air within the housing.

Ideally, the means for sterilising air within the housing is configurable to remove particulate matter from the air within the housing via the induction of counter current flow between a fluid and the air within the housing.

Preferably, the fluid supply means is configurable to supply droplets of fluid to air within the housing.

Preferably, the fluid is a solvent configurable to absorb and/or entrap particulate matter suspended in the air within the housing.

Preferably, the fluid is a liquid.

Ideally, the fluid comprises water. Ideally, the fluid comprises less than 9% hydrogen peroxide. Ideally, the fluid comprises less than 8%, less than 7%, less than 6%, less than 5%, less than 4% or less than 3% hydrogen peroxide.

Ideally, the fluid and/or droplets are configured to absorb and/or entrap particulate matter in the air within the housing.

Advantageously, this causes separation of the particulate matter from the air.

Preferably, the fluid and/or droplets are configured to remove particulate matter from the air within the housing.

Preferably, the replaceable cartridge comprises a concentration of hydrogen peroxide less than 9%.

Ideally, the replaceable cartridge comprises a concentration of hydrogen peroxide less than 8%, preferably less than 7%, preferably less than 6%, preferably less than 5%, preferably less than 4%, preferably less than 3%.

Ideally, the replaceable cartridge has a means for releasably coupling the cartridge at the base of the air sterilisation unit.

Preferably, the replaceable cartridge has a means for releasably mechanically coupling the cartridge at the base of the air sterilisation unit.

Ideally, the means for releasably coupling the cartridge at the base of the air sterilisation unit comprises magnetic means.

Preferably, the mechanical coupling means comprises screw fit means, press fit means, snap fit means, interference fit means or any other suitable detachable mechanical coupling means disposed between the cartridge and the air sterilisation unit.

Preferably, the means for delivering the means for sterilising air into the air sterilisation unit comprises the wick.

Ideally, the replaceable cartridge has a piezo pump.

Preferably, the means for delivering the means for sterilising air into the air sterilisation unit comprises the piezo pump.

Preferably, the air sterilisation unit comprises a means for preventing UV light from escaping the air sterilisation unit.

Ideally, the means for preventing UV light from escaping the air sterilisation unit prevents at least some, most preferably most, of the UV light from escaping the air sterilisation unit via the air outlet means.

Ideally, the means for preventing UV light from escaping the air sterilisation unit comprises a protective layer.

Ideally, the protective layer is located between the one or more UV lamps and the air outlet means.

Ideally, the protective layer permits movement of air across the layer but restricts movement of UV light across the layer.

Preferably, the air sterilisation unit comprises porous media configured to receive hydrogen peroxide from the hydrogen peroxide reservoir.

Ideally, the porous media is located in the air outlet chamber, and/or between the droplet separator and air outlet means. The porous media may be formed from ceramic or fibrous material.

Advantageously, the porous media can retain a quantity of hydrogen peroxide on or within the media. Air moves through the porous media towards the air outlet means and this generates hydrogen peroxide vapour in the air outlet chamber.

In use, the hydrogen peroxide from the reservoir is transferred onto the porous media and/or into the air outlet chamber. The hydrogen peroxide is in a fluid form but is converted into the vapour by the movement of air through the air sterilisation unit. The hydrogen peroxide vapour then reacts with the UV light from the UV lamps. This in turn kills/deactivates 99.9% of pathogens and organic compounds in a single air pass.

In a further embodiment, the air sterilisation unit comprises one or more filters located between the air intake means and the air outlet means.

Preferably, the one or more filters are located closer to the air intake means than the air outlet means.

Ideally, the air that enters the air sterilisation unit via the air intake means contacts a filter before any other component of the air sterilisation unit in this embodiment.

Ideally, the one or more filters comprise a filter for large dust extraction, and/or a carbon filter.

According to a second aspect of the invention there is provided an air heating, ventilation, conditioning and/or sterilisation system having one or more air sterilisation units comprising: a housing; an air intake means for allowing air to enter the housing; an air outlet means for allowing air to exit the housing; and a means for sterilising air within the housing.

Preferably, the system is a continuous heated and conditioned air system.

The air sterilisation unit of the system may have any of the features of the air sterilisation unit according to the first aspect of the invention and in any combination.

The air outlet means of a first air sterilisation unit may be directly or indirectly connected to the air intake means of a second air sterilisation unit such that air that passes initially through the first air sterilisation unit then passes through the second air sterilisation unit before being released.

According to a third aspect of the invention there is provided a method for sterilising air, the method comprising: generating a flow of air and generating a flow of fluid wherein at least part of the flow of fluid is countercurrent to the flow of air.

Preferably the method comprises generating a flow of air within an air sterilisation unit.

The air sterilisation unit of the system may have any of the features of the air sterilisation unit according to the first aspect of the invention and in any combination.

Preferably, the method comprises generating a flow of fluid within the air sterilisation unit.

Preferably, the method comprises generating a flow of fluid in the form of droplets within the air sterilisation unit.

Preferably, the method comprises generating a flow of sterilised fluid within the air sterilisation unit that is countercurrent to the flow of air.

Preferably the method comprises generating a flow of air within a housing.

Preferably the method comprises generating a flow of fluid within a housing.

Preferably, the method comprises mixing of the fluid and the air and/or inducing contact between the fluid and the air.

Advantageously, any particulate matter and/or carbon dioxide within the air will be entrapped and/or removed from the air by the fluid.

Preferably, the method comprising the step of confining the fluid to the air sterilisation unit.

Preferably the method comprises generating a flow of air through a plurality of chambers within the housing.

Preferably, the method comprises the step of generating fluid flow through a plurality of chambers.

Preferably, the method comprises generating a flow of air through the air sterilisation unit.

Preferably, the method comprises generating a flow of air through an air sterilisation chamber.

Preferably, the method comprises generating a flow of liquid through an air sterilisation chamber.

Preferably the method comprises generating a flow of air into the air sterilisation unit.

Preferably the method comprises using air flow generation means to generate a flow of air into, through, within and/or out of an air sterilisation unit.

Preferably the method comprises drawing un-sterilised air into the air sterilisation unit.

Preferably, the method comprises the step of exposing the air to one or more means of sterilising air within the housing.

Preferably, the method comprises the step of exposing the air to sterilised fluid.

Preferably, the method comprises the step of activating one or more means of sterilising air within the housing.

Preferably, the method comprises generating air flow and counter current fluid flow through a scrubbing tower and/or packing material.

Advantageously, air within the housing is sterilised via fluid scrubbing. Further advantageously, the speed of air and/or fluid flow is reduced. Thereby, fluid-particle and/or fluid-air contact is increased.

Preferably, the method comprises generating air flow through a droplet/liquid separator.

Advantageously, this prevents droplets or fluid entrained in the air flow from escaping the air sterilisation unit. Thereby, this prevents particulate matter from escaping the air sterilisation unit.

Preferably, the method comprises generating air flow through an air outlet chamber.

Preferably, the method comprises exposing the air flow to one or more UV lamps to further deactivate and/or remove hydrogen peroxide residue from the air flow.

Preferably, the method comprises the step of exposing the air to one or more humidifiers in the air outlet chamber.

Advantageously, this removes any excess or unwanted moisture from the air prior to its exhaustion from the air sterilisation unit.

Preferably, the method comprises collecting fluid in a fluid reservoir.

Preferably, the method comprises generating a flow of fluid through a fluid disinfection chamber or reservoir.

Preferably, the method comprises the step of generating a flow of fluid into, through and/or out of the fluid reservoir.

Preferably, the method comprises collecting used and/or un-sterilised fluid in the fluid reservoir.

Preferably, the method comprises the step of generating a flow of used and/or un-sterilised fluid into the fluid reservoir.

Preferably, the method comprising the step of sterilising fluid in the fluid reservoir and/or as fluid flows through the fluid reservoir.

Preferably, the method comprises the step of exposing fluid to one or more fluid sterilising means.

Preferably, the method comprises the step of exposing fluid to one or more fluid sterilising means, prior to the generation of the flow of fluid.

Preferably, the method comprises exposing un-sterilised fluid and/or fluid containing particulate matter to one or more fluid sterilisation means.

Preferably, the method comprises activation of one or more fluid sterilisation means.

Preferably, the method comprises generating a sterilisation agent and/or supplying a sterilisation agent to fluid in the fluid reservoir. Advantageously, the sterilisation agent can sterilise and/or deactivate particulate matter within the fluid in the reservoir.

Preferably, the method comprises using a reactor to generate a sterilisation agent.

Preferably, the method comprises generating hydrogen peroxide and/or singlet oxygen.

Preferably, the method comprises generating a sterilisation agent from water and/or one or more other base materials.

Preferably, the method comprises generating a sterilisation agent using electrolysis.

Preferably, the method comprises generating a sterilisation agent using water electrolysis.

Preferably, the method comprises generating a sterilisation agent using a photodynamic reaction.

Preferably, the method comprises generating a sterilisation agent by exposing one or more of the base materials to a light source.

Preferably, the method comprises exposing the fluid to the sterilisation agent.

Preferably, the method comprising the step of generating a flow of sterilised fluid out of the fluid reservoir for re-use in the sterilisation of air within the housing.

Preferably, the method comprises preventing the sterilisation agent from escaping the air sterilisation unit.

Preferably the method comprises confining the sterilisation agent to the air sterilisation unit and/or housing. Advantageously, confining the sterilisation agent means that the sterilisation agent will not contaminate surrounding areas such as where persons may be present.

Preferably, the method comprises allowing sterilised air to leave the air sterilisation unit.

Preferably, the method comprises removing sterilisation agent from at least some of the fluid within the air sterilisation unit.

Preferably, the method comprises exhausting air from the air sterilisation unit.

Preferably, the method comprises exhausting sterilised air from the air sterilisation unit or space.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

The invention will now be described with reference to the accompanying drawings in which:

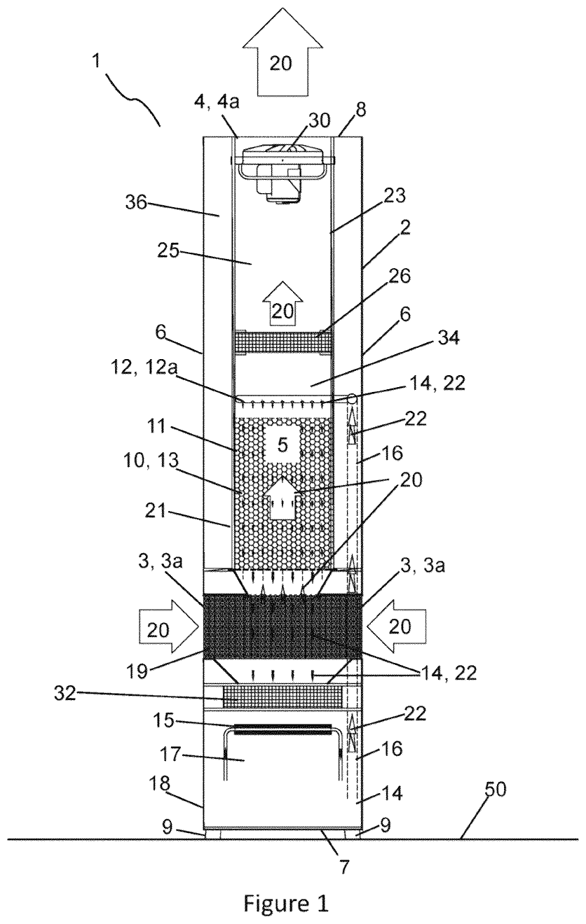

FIG. 1 is a cross sectional view of an air sterilisation unit according to an aspect of the invention.

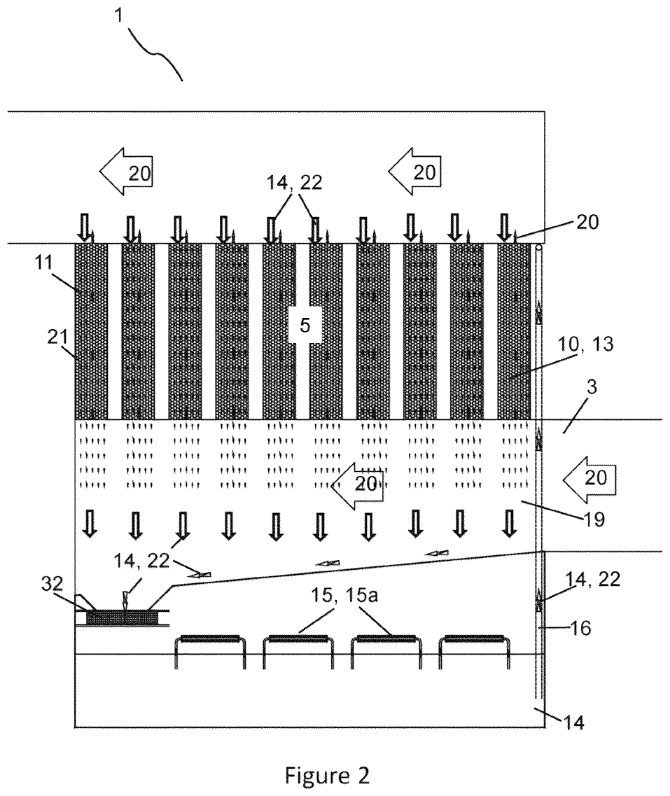

FIG. 2 is a section view of an air sterilisation system according to a second aspect of the invention.

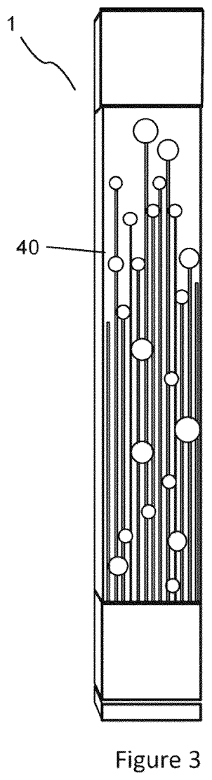

FIG. 3 is a perspective view of the sterilisation unit comprising a cover member.

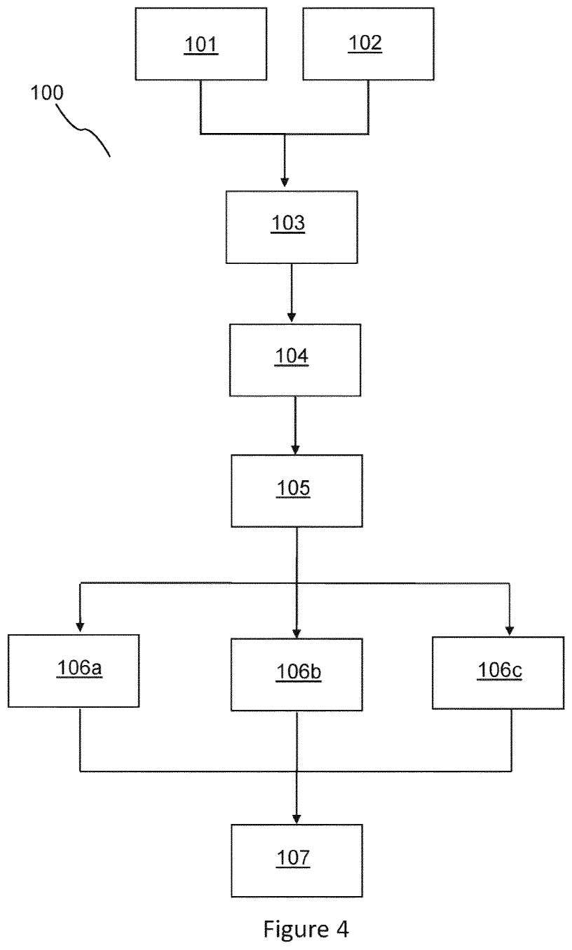

FIG. 4 is a schematic view of a method according to an aspect of the invention.

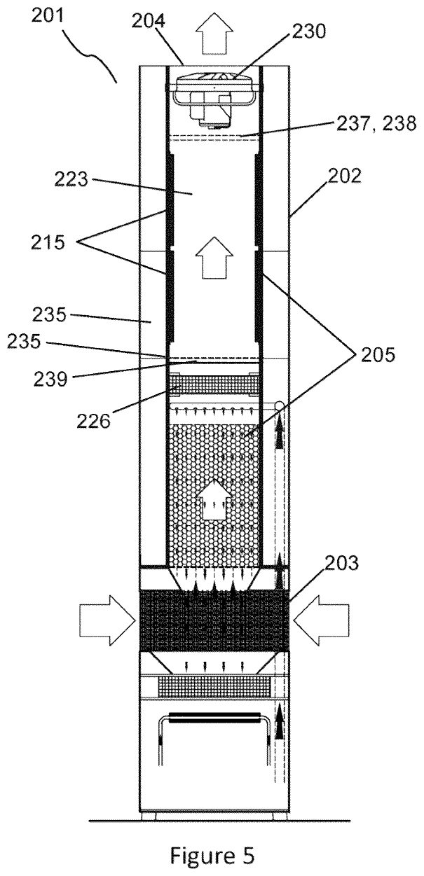

FIG. 5 is a cross sectional view of a further embodiment of an air sterilisation unit according to an aspect of the invention.

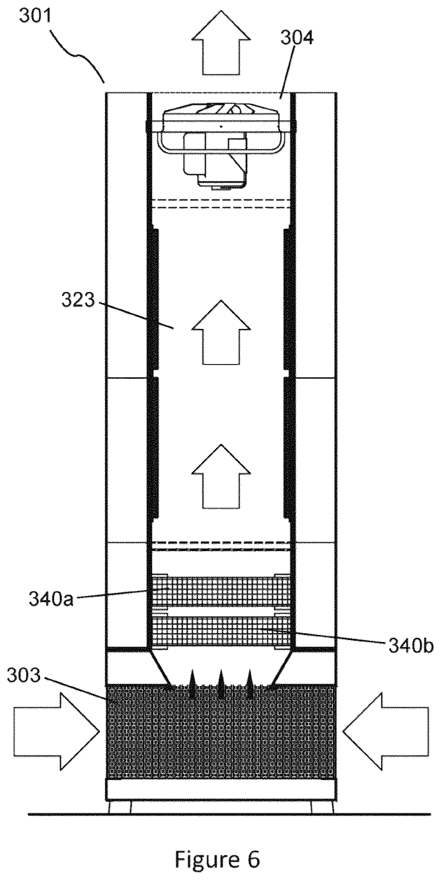

FIG. 6 is a cross sectional view of yet a further embodiment of an air sterilisation unit according to an aspect of the invention.

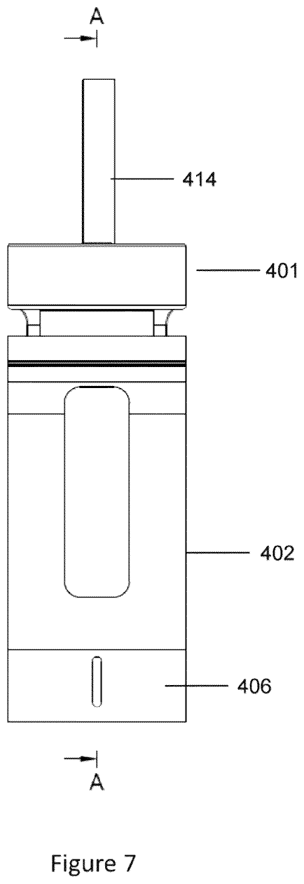

FIG. 7 is an exterior perspective view of a further embodiment of the present invention.

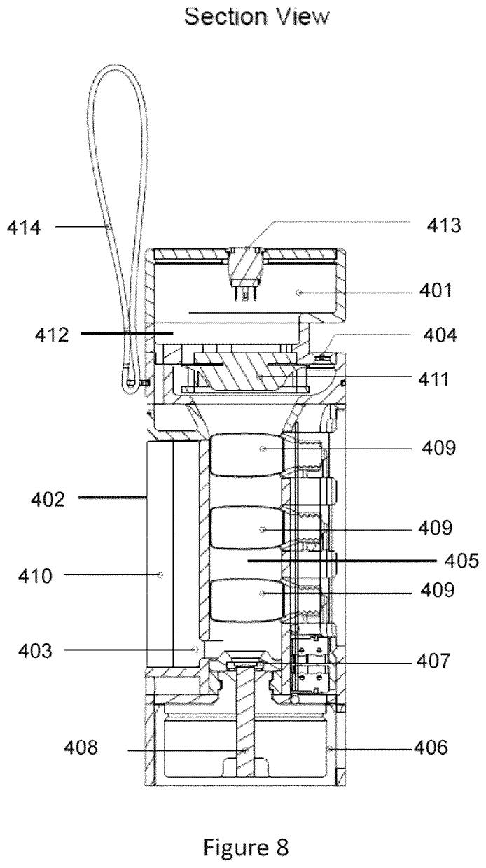

FIG. 8 is a cross section view displaying the interior of the embodiment of FIG. 7.

In FIG. 1 there is shown an air sterilisation unit 1 according to an embodiment of the invention. The air sterilisation unit 1 comprises a housing 2, an air intake arrangement 3 for allowing air to enter the housing 2, an air outlet arrangement 4 for allowing air to exit the housing 2, and an arrangement 5 for sterilising air within the housing 2. The air sterilisation unit 1 allows contaminated air to enter the housing 2, can sterilise air by entrapping or removing particulate matter including deactivating airborne micro-organisms viruses, bacteria, dust, pathogens, VOCs and/or any other particulate matter present in the air inside the housing 2 and allows clean air to leave the housing 2.

As will be explained in further detail below, sterilisation of the air is confined to the air sterilisation unit 1.

The present embodiment of the air sterilisation unit 1 is a tower having side walls 6, a lower member or base 7 and an upper member or lid 8. The or each side wall 6 extends between the lower member or base 7 and the upper member or lid 8. The air sterilisation unit 1 comprises support arrangements in the form of legs 9 which are adapted to support the housing in the upright position on a surface 50. The legs 9 are adapted to hold the air sterilisation unit 1, and particularly the air intake arrangement 3, at an appropriate height off the ground. Although two legs 9 are shown in the cross-sectional view of FIG. 1 it will be understood that the air sterilisation unit may comprise any number of legs 9 to provide a suitable and sturdy support base. The support legs 9 are attached to the lower member or base 7 of the housing 2.

The air intake arrangement 3 comprises a plurality of apertures 3a adapted to allow air to pass into the housing 2. The housing 2 comprises an air intake chamber 19. The air intake arrangement 3 is located in a side wall 6 of the housing 2, in particular in a lower portion of the housing side wall 6 adjacent to the air intake chamber 19. The plurality of intake apertures 3a surround a portion of the housing side wall 6, allowing the air sterilisation unit 1, and particularly the air intake arrangement 3, to draw air into the air sterilisation unit 1 from a plurality of directions. Air is able to enter the arrangement 5 for sterilising air within the housing 2 via the air intake arrangement 3. The air outlet arrangement 4 comprises an outlet aperture 4a adapted to allow air to pass out of the housing 2. The air outlet arrangement 4 is located in the upper member or lid 8 of the housing 2. The air sterilisation unit 1 comprises an air flow path 20. The air intake arrangement 3 is in fluid communication with the air outlet arrangement 4 via the air flow path 20. Air is able to flow through the housing 2 via the air flow path 20.

The air sterilisation unit 1 comprises an air flow generation arrangement 30. The air flow generation arrangement 30 is adapted to cause air to flow into, through and out of the housing 2. The air flow generation arrangement 30 is used to create a strong, continuous flow of air along the air flow path 20 and through the unit 1. In use, the air flow generation arrangement 30 is activated to draw un-sterilised air into the air sterilisation unit 1 where said air will be sterilised by the arrangement 5 for sterilising air within the housing 2. It will be understood that a plurality of air flow generation arrangements may be used. Each air flow generation arrangement 30 has a fan and a motor. The air flow generation arrangement 30 is adapted to generate a fluid flow of air along the air flow path 20. The air flow generation arrangement 30 is adapted to generate a fluid flow of air in a direction from the air inlet arrangement 3 towards the air outlet arrangement 4.

The arrangement 5 for sterilising air within the housing 2 is configured to entrap and/or remove particulate matter and/or carbon dioxide from air within the housing 2. This prevents contaminated air from being dumped back into the environment surrounding the air sterilisation unit 1. This is in comparison to air filtration units or HVAC systems which rely on filters to remove particulate matter from the air. By particulate matter we mean airborne micro-organisms, viruses, bacteria, dust, pathogens, VOCs and/or any other undesirable matter suspended in the airflow.

The arrangement 5 for sterilising air within the housing 2 comprises a fluid supply arrangement 12 for supplying fluid 14 to air within the housing 2. The arrangement 5 for sterilising air within the housing 2 has a fluid supply arrangement 12 for supplying fluid 14 to air within the housing in a direction that is counter current to the flow 20 of air within the housing 2. The arrangement 5 for sterilising air within the housing 2 is capable of removing particulate matter from the air within the housing 2 via the induction of counter current flow between a fluid and the air the housing.

The fluid supply arrangement is adapted to generate a flow of fluid within the air sterilisation chamber 21. The fluid supply arrangement 12 is adapted to supply droplets of fluid 14 to air within the housing 2. The fluid 14 is a solvent capable of absorbing and/or entrapping particulate matter from the air within the housing 2. In the embodiment illustrated, the fluid comprises less than 9% hydrogen peroxide. The fluid and/or droplets are capable of absorbing and/or entrapping particulate matter from the air within the housing 2. This causes separation of the particulate matter form the air. The fluid and/or droplets are capable of removing particulate matter from the air within the housing 2. The air sterilisation unit 1 has a fluid delivery arrangement 16. The fluid delivery arrangement 16 is adapted to supply fluid 14 from one or more parts of the air sterilisation unit 1 to one or more other parts of the air sterilisation unit 1. In particular, the fluid delivery arrangement 16 is adapted to deliver fluid from one or more parts of the air sterilisation unit 1 to the fluid supply arrangement 12. The arrangement 5 for sterilising air within the housing has an arrangement 10 for manipulating fluid-particle contact within the housing. The arrangement 5 for sterilising air within the housing 2 has an arrangement 10 for increasing fluid-particle contact within the housing. The arrangement 10 for manipulating fluid-particle contact within the housing is adapted to increase fluid-particle contact within the housing 2. The arrangement 10 for manipulating fluid-particle contact within the housing 2 is adapted to manipulate the speed and/or direction of airflow and/or fluid flow through the housing 2. The arrangement 10 for manipulating fluid-particle contact within the housing is adapted to reduce the speed of airflow and/or fluid flow through the housing 2. This increases fluid-particle and/or solvent-particle contact. Thereby, this increases the likelihood of separation of particulate matter and/or carbon dioxide from the air within the housing. Further, this means that air and particulate matter and/or carbon dioxide within the housing 2 will be exposed to fluid for a longer period of time. This is in comparison to not having an arrangement 10 for manipulating fluid-particle contact within the housing.

The air sterilisation unit 1 is adapted to perform scrubbing of air to sterilise the air and/or to remove particulate matter and/or carbon dioxide from the air. The unit 1 or arrangement 5 for sterilising air within the housing 2 comprises a scrubbing tower 11. The scrubbing tower 11 is a fluid scrubbing tower. Therefore, air sterilisation unit 1 is adapted to perform fluid scrubbing of air to provide air sterilisation. This provides an environmental solution to air sterilisation via the scrubbing tower 11. The air sterilisation unit 1 or scrubbing tower 11 comprises packing material 13.

The arrangement 10 for manipulating fluid-particle contact within the housing is or comprises packing material 13. The packing material 13 is adapted to manipulate the speed and/or direction of airflow 20 or fluid flow 22 through the housing 2. The packing material 13 is adapted to to slow airflow through the housing 2 such that fluid-particle contact and/or solvent-particle contact is increased. The packing material 13 can be configured to slow airflow 20 and/or fluid flow 22 through the housing 2 such that fluid-particle contact and/or solvent-particle contact is increased. The packing material 13 provides an elongated path for air flow 20 and/or fluid flow 22 through the housing 2. Having an elongated path means that the air and/or fluid within the housing 2 takes longer to pass through the housing 2, and therefore the air will be exposed to the fluid for an increased amount of time. Thereby, this increases contact between the air and fluid to ensure adequate removal of particulate matter and/or carbon dioxide from the air.

The air sterilisation unit 1 comprises a fluid reservoir 18 configurable to contain fluid 14. The fluid delivery arrangement 16 is adapted to deliver fluid or droplets of fluid 14 to the arrangement 5 for sterilising air within the housing 2. The fluid 14 and/or droplets are capable of carrying particulate matter to the fluid reservoir 18 via gravity. The fluid reservoir 18 comprises a fluid sterilisation arrangement 15. The fluid sterilisation arrangement 15 is adapted to sterilise and/or disinfect fluid 14 within the fluid reservoir 18. This means that fluid containing particulate matter can be sterilised for re-use. The fluid sterilisation arrangement 15 comprises one or more aquatic UV lamps 15a adapted to sterilise and/or disinfect the fluid 14. After sterilisation and/or disinfection, the fluid 14 can be fed back into the air sterilisation arrangement 5 for re-use. Therefore, this reduces the amount of fluid being consumed by the air sterilisation unit 1. Further, this reduces costs and operator involvement in relation to the management of the air sterilisation unit 1.

The fluid sterilisation arrangement 15 comprises a sterilisation agent. The fluid sterilisation arrangement 15 comprises an arrangement for supplying sterilisation agent. The arrangement for supplying a sterilisation agent is located within the housing 2. The arrangement for supplying a sterilisation agent is located within the fluid reservoir 18. The arrangement for generating sterilisation agent is adapted for continuous production of sterilisation agent. The fluid sterilisation arrangement 15 has multiple arrangements for generating one or more sterilisation agents. The arrangement for generating sterilisation agent is adapted for continuous production of hydrogen peroxide and/or singlet oxygen. The arrangement for generating sterilisation agent continuously produces sterilisation agent, in use. The fluid sterilisation arrangement 15 comprises a hydrogen peroxide reactor (not illustrated). The hydrogen peroxide reactor is adapted to produce hydrogen peroxide from one or more base materials such as hydrogen, oxygen and water. The sterilisation agent is hydrogen peroxide.

Additionally, or alternatively, the sterilisation agent is or comprises singlet oxygen. The fluid sterilisation arrangement comprises a singlet oxygen generator. The fluid sterilisation arrangement 15 is adapted to sterilise fluid in the reservoir 18 via one or more photodynamic reactions and/or singlet oxygen producing reactions. The fluid sterilisation arrangement 15 has one or more base materials and/or reactants capable of reacting to produce singlet oxygen. The fluid sterilisation arrangement comprises an activation arrangement adapted to activate the one or more base materials and/or a photodynamic reaction. The activation arrangement comprises a light source. The activation arrangement comprises one or more LEDs and/or UV lamps.

The fluid sterilisation arrangement 15 comprises a base material reservoir. The base material reservoir is able to store a ready supply of the base material(s) used to make the sterilisation agent. The fluid sterilisation arrangement comprises a sterilisation agent reservoir. The sterilisation agent reservoir is able to store a ready supply of the sterilisation agent for use within the fluid sterilisation means and/or fluid reservoir. The sterilisation agent is locatable within the sterilisation agent reservoir. The arrangement for supplying a sterilisation agent is adapted to output sterilisation agent to the sterilisation agent reservoir, such as via a tube or conduit. The sterilisation agent is capable of sterilising and/or disinfecting fluid 14 within the fluid reservoir 18. Therefore, sterilisation is maintained within the housing.

The air sterilisation unit 1 has one or more heaters 17 adapted to heat the fluid 14 and/or water 14 within the fluid reservoir 18.

The air sterilisation unit 1 comprises a further arrangement 25 for sterilising air within the housing 2. The further arrangement 25 for sterilising air within the housing 2 has one or more UV lamps (not illustrated) adapted to sterilise air within the housing 2.

The air sterilisation unit 1 is portable. The air sterilisation unit 1 has an air sterilisation chamber 21. The arrangement 5 for sterilising air within the housing 2 is located within the air sterilisation chamber 21. The housing 2 comprises an air intake chamber 19. Air is able to enter the air intake chamber 19 via the air intake arrangement 3.

The arrangement 5 for sterilising air within the housing 2 is located in a middle or lower portion of the housing 2. The air sterilisation unit 1 comprises an air outlet chamber 23. The air outlet arrangement 4 is located adjacent to the air outlet chamber 23. The arrangement 5 for sterilising air within the housing 2 is located between the air intake arrangement 3 and the air outlet arrangement 4. The air intake arrangement 3 is located adjacent to a lowermost portion of the air sterilisation chamber 21. The air intake arrangement 3 is in fluid communication with the air outlet arrangement 4 via the air flow path 20. The air flow path 20 is located entirely or at least partially within the housing 2. The air flow path 20 is located at least partially within the arrangement 5 for sterilising air within the housing 2. The air flow path 20 is located at least partially within the air sterilisation chamber 21. This ensures that air flows through the air sterilisation chamber 21 and/or is exposed to the arrangement 5 for sterilising air within the housing 2 so that it can be sterilised.

The air sterilisation unit 1 has droplet separator 26. The droplet separator 26 is adapted to separate any fluid 14 entrained in the air within the housing 2. This prevents the escape of any particulate matter and/or hydrogen peroxide that has been captured by has been captured by the fluid 14 from escaping the air sterilisation unit 1 via fluid entrained in the air. The droplet separator 26 is located between the air sterilisation chamber 21 and the air outlet chamber 23.

The further arrangement 25 for sterilising air within the housing 2 is located within the air outlet chamber 23. The air sterilisation unit 1 has a dehumidifier (not illustrated). Any unwanted moisture or fluid will be removed by the dehumidifier. Further, this prevents particulate matter and/or hydrogen peroxide that has been captured by the fluid 14 from escaping the air sterilisation unit 1. The dehumidifier is located above the fluid supply arrangement 12. This prevents fluid from the fluid supply arrangement, intended to flow through the air sterilisation chamber 21, from being removed as soon as it exits the fluid supply arrangement 12. The dehumidifier is located above the air sterilisation chamber 21 and/or arrangement 5 for sterilising air within the housing 2. The dehumidifier is located within the air outlet chamber 23. This means that any excess moisture in the air flowing through the air outlet chamber 23 will be removed prior to the air exiting the air sterilisation unit 1 via the air outlet arrangement 4. The dehumidifier is located above the droplet separator 26. Therefore, any fluid or moisture which manages to bypass the droplet separator 26 will be exposed to the dehumidifier.

In use, air is able to enter the air sterilisation unit 1 via the air intake arrangement 3, travel through the air sterilisation chamber 21, through the droplet separator 26, through the air outlet chamber 23 and out of the air outlet arrangement 4.

The air sterilisation unit 1 comprises a fluid flow path 22. Fluid 14 is able to flow through the housing 2 via the fluid flow path 22. The fluid flow path 22 is located entirely within the housing 2. The fluid flow path 22 is located at least partially within the arrangement 5 for sterilising air within the housing 2. This ensures exposure of the air flow path 20 to the fluid flow path 22. Further, this ensures contact between the fluid 14 in the fluid flow path and air and/or particulate matter or carbon dioxide in the air flow path 20. The fluid flow path 22 runs countercurrent to the air flow path 20. In this embodiment, the air flow path, generated by the air flow generation arrangement 30, runs upwardly from the air intake arrangement 3 to the air outlet arrangement 4 and at least part of the fluid flow path 22 runs downwardly via gravity from the fluid supply arrangement 12 to the fluid reservoir 18. At least part of the fluid flow path 22 also runs from the fluid reservoir upwardly, via the fluid delivery arrangement 16, to the fluid supply arrangement 12. Delivery of the fluid 14 from the reservoir 18 to the fluid supply arrangement 12 occurs in an outer chamber of the air sterilisation unit and/or in a conduit which is separated from the air flow pathway 20. This means that the airflow 20 is only exposed to the downward, countercurrent, flow of fluid.

The fluid supply arrangement 12 is in fluid communication with the fluid reservoir 18 via the fluid flow path 22. The fluid supply arrangement has one or more fluid outlet apertures 12a. The one or more fluid outlet apertures 12a are located at an uppermost portion of the air sterilisation chamber 21 and/or of the scrubbing tower 11. The fluid supply arrangement 12 is located above the arrangement 10 for manipulating fluid-particle contact within the housing and/or above the packing material 13. The fluid reservoir 18 is located below the air sterilisation chamber 21 and/or the scrubbing tower 11. The fluid reservoir is 18 located below the arrangement 10 for manipulating fluid-particle contact within the housing 2 and/or the packing material 13. This means that fluid 14 from the fluid supply arrangement 12 can flow from the fluid outlet aperture(s) 12a through the housing 2, air sterilisation chamber 21 and/or is exposed to the arrangement 5 for sterilising air within the housing 2 via gravity to the fluid reservoir 18. The fluid reservoir 18 is located below the air intake arrangement 3 and/or air intake chamber 19. Therefore, fluid 14 flows through the air intake chamber 19 to begin sterilising air as soon as the air enters the housing 2.

The fluid delivery arrangement 16 is adapted to deliver sterilised fluid 14 from the fluid reservoir 18 to the fluid supply arrangement 12 and/or one or more fluid outlet apertures 12a.

The air flow generation arrangement 30 is adapted to cause air to flow into the air sterilisation unit 1 via the air intake arrangement 3. The air flow generation arrangement 30 is adapted to allow air to flow through the air flow generation arrangement 30. The air flow generation arrangement 30 is adapted to cause air to flow into and/or through the housing 2 where it is exposed to the arrangement 5 for sterilising air within the housing 2. Un-sterilised air can be drawn into the air sterilisation unit 1 by the air flow generation arrangement 30 so that said air can be sterilised. The air flow generation arrangement 30 is located within the housing 2. The air flow generation arrangement 30 is adapted to cause air to flow into, through and/or out of the air outlet chamber 23. The air flow generation arrangement 30 is adapted to cause air to flow out of the air sterilisation unit 1 via the air outlet arrangement 4. The air flow generation arrangement 30 is adapted to cause air to flow out of the housing 2 and/or air sterilisation chamber 21. The air flow generation arrangement 30 is located within or adjacent to the air outlet chamber 23. The air flow generation arrangement 30 is adjacent to the air outlet arrangement 4. Only one air glow generation arrangement is illustrated in the drawings, however, it will be understood that the air sterilisation unit 1 can have any number of air flow generation arrangements. The air flow generation arrangement 30 is adapted to provide a continuous flow of air without interrupting the countercurrent flow of fluid. The or each air flow generation arrangement 30 comprises a fan. The or each air flow generation arrangement 30 comprises a motor.

The air sterilisation unit 1 has a filtration arrangement. The air sterilisation unit 1 has a fluid filtration arrangement 32. The fluid filtration arrangement 32 can remove particulate matter and/or sediment from fluid 14 travelling into, through, within and/or out of the fluid reservoir 18. The fluid filtration arrangement 32 is removable and/or replaceable. Therefore, used and/or clogged fluid filtration arrangement 32 can be replaced with new and pristine fluid filtration arrangement, providing better filtration.

The air sterilisation unit 1 has an air filtration arrangement 40 so that fine particles such as dust and pollen travelling into, through, within and/or out of the air sterilisation unit 1 can be removed from the air. The air sterilisation unit 1 has an outer air filtration arrangement 40 adapted to filter particles such as dust and/or pollen at the point of entry 19 into the air sterilisation unit 1. The outer air filtration arrangement 40 comprises a cover for the air sterilisation unit 1. The cover is a fabric or synthetic material cover arrangement. The cover comprises indicia.

The air sterilisation unit 1 may also have one or more further air filtration arrangements such as an intake air filtration means locatable adjacent to the air inlet arrangement 3. The intake air filtration arrangement is adapted to filter particles such as dust and/or pollen within the air sterilisation unit 1. The intake air filtration arrangement is adapted to filter particles such as dust and/or pollen moving from the intake chamber 19 towards the air sterilisation chamber 21. The intake air filtration arrangement has one or more filters locatable adjacent to the air intake arrangement 3. The air sterilisation unit 1 may also have an outlet air filtration arrangement locatable between the air flow generation arrangement 30 and the air outlet arrangement 4. The outlet air filtration arrangement has a vapour control layer. The outlet air filtration arrangement has a layer of fabric or synthetic material. The outlet air filtration arrangement has a layer of felt. The outlet air filtration arrangement has a layer of air-permeable, fluid-impermeable material. The or each air filtration arrangement comprises at least one filter such as a HEPA filter.

Water and sterilisation agent within the unit is recycled. The air sterilisation unit 1 may have one or more sensors to measure one or more operating conditions of the unit 1. For example, the air sterilisation unit 1 may have one or more sensors for detecting any escape of sterilisation agent from the air sterilisation unit 1. Sterilisation agent sensors may be located adjacent to the outlet arrangement 4. In the event of detecting sterilisation agent outside the air sterilisation unit 1, the sterilisation unit 1 may be configured to shut down and/or send a notification to a central server or hub. The air sterilisation unit 1 may include sensors for detecting the concentration of sterilisation agent in the sterilisation agent reservoir and/or the concentration of sterilisation agent being produced by the arrangement for generating sterilisation agent. In the event of detecting a concentration outside a desired or set parameter (for example, more than 1 PPM), the sterilisation unit 1 may be configured to shut down and/or send a notification to a central server or hub. The air sterilisation unit 1 may comprise a communication arrangement such as wireless communication arrangement that is connectable to a central hub or server. The communication arrangement may be adapted to send sensor data and/or performance information. The air sterilisation unit 1 may be adapted to send and/or receive notifications.

The air sterilisation unit 1 has an inner chamber 34 and an outer chamber 36. The outer chamber 36 surrounds the inner chamber 34. The outer chamber 36 is adapted to house one or more controls and/or one or more other parts of the air sterilisation unit 1. At least part of the fluid delivery arrangement 12 is located within the outer chamber 36. The air sterilisation unit 1 is a closed loop air sterilisation unit 1. By ‘closed-loop’, we mean that sterilisation of the air is maintained within the air sterilisation unit 1. This prevents escape of fluid 14, particulate matter, carbon dioxide and/or sterilisation agent from the air sterilisation unit 1. The air sterilisation unit 1 is adapted to perform cyclic sterilisation of air. The air sterilisation unit 1 is adapted to provide up to 1000 sq/ft with sterilised and/or conditioned air approximately or at least eight time per hour. Air sterilisation unit 1 forms part of a heat recovery system. The air sterilisation unit 1 is adapted to perform heat recovery. The air sterilisation unit 1 can is operable with one or more air sterilisation units 1. Therefore, multiple air sterilisation units 1 can be grouped to and/or added to large HVAC systems to replace heat recovery units, recirculating air in a building by returning sterilised and/or conditioned air via a closed system. Further, multiple air sterilisation units 1 can be grouped to regularly or irregularly provide sterilised and/or conditioned air to areas larger than 1000 sq/ft.

In FIG. 4 there is shown a method 100 for sterilising air according to an embodiment of the invention. While in the example embodiment the air sterilisation unit 1 is used to carry out the method 100, it will be appreciated that the method 100 can be carried out using the air sterilisation unit 1 or any alternative apparatus having all of the necessary features for carrying out the method 100.

The method 100 comprises: generating a flow of air in step 101; and generating a flow of fluid wherein at least part of the flow of fluid is countercurrent to the flow of air 102. The fluid is able to entrap and/or remove all particulate matter present in the air. As will be appreciated, any or all of the steps of the method 100 may be carried out sequential, simultaneously and/or may be carried out continuously.

In step 101 a flow of air is created, for example a flow of air within the air sterilisation unit 1, particularly within the housing 2. The example method 100 comprises generating a flow of air through the plurality of chambers 19, 21 and 23 within the housing 2. The air flow generation arrangement 30 can be activated to generate a flow of air into, through, within and out of the air sterilisation unit 1. Un-sterilised air is drawn into the air sterilisation unit 1 via the air intake arrangement 3.

Step 102 comprises generating a flow of fluid by activating the fluid supply arrangement 12 and the fluid delivery arrangement 16. For example, fluid is supplied by the fluid supply arrangement 12 and travels through the housing via gravity towards the reservoir 18. Sequentially, or simultaneously, sterilised fluid is delivered from the reservoir 18 via the fluid delivery arrangement 16 towards the fluid supply arrangement 12.

Step 103 refers to exposing the air to the sterilised fluid by generating the air flow path 20 and fluid flow path 22 through the same part of the housing 2 at the same time. For example, generating air flow 20 and fluid flow 22 through the air sterilisation chamber 21 and through, over or between the packing material 13 which causes an elongation of the path 20 and 22 and thereby increases contact between the fluid and the air.

Step 104 refers generating air flow through the air outlet chamber 23 after the air has been in contact with sterilised fluid. Air flow 20 through the air outlet chamber 23 is generated via the airflow generation arrangement 30. In the air outlet chamber 23, the air may also be exposed to a droplet separator 26 and a further arrangement 25 for sterilising air within the housing 2 such as one or more UV lamps. The air may also be exposed to humidifiers which removes any excess or unwanted moisture from the air.

In step 105 the method 100 comprises exhausting sterilised air from the air sterilisation unit 1.

Step 106a-106c comprises sterilising the fluid in the fluid reservoir 18. Sterilisation of the fluid can be initiated by activation of the fluid sterilisation arrangement 15. For example, 104a relates to activating one or more UV lamps/sterilizers 15a to deactivate particulate matter within the fluid and/or to sterilise the fluid. Further, 104b refers to exposing one or more base materials to a light source or other stimulant or reactant to activate a singlet oxygen producing reaction or photodynamic reaction and outputting the singlet oxygen to the reservoir 18. Further, 104c refers to the generation of a sterilisation agent, for example using a reactor to generate hydrogen peroxide from water using electrolysis, outputting hydrogen peroxide to the reservoir 18. Steps 103a-103c may be carried out simultaneously, sequentially or not at all.

In step 107 the fluid and any sterilisation agent is confined to the air sterilisation unit 1 by preventing escape of the fluid and any sterilisation agent from escaping the air sterilisation unit 1. Confining the fluid and any sterilisation agent to the enclosed volume within the air sterilisation unit 1 means that neither will contaminate surrounding areas such as where persons may be present. This enables a closed-loop system and/or re-use of the fluid in sequential/continuous air sterilisation.

As will be understood by the skilled person, the example embodiments presented above can be modified in a number of ways without departing from the scope of the invention. For example, the air sterilisation unit 1 and/or method 100 can be used in a wide variety of areas and situations such as in workplaces, shops, restaurants, schools, homes or hospitals. Where a person is clinically vulnerable the air sterilisation unit 1, which is a portable stand-alone unit, can be deployed in a room in which the person is undergoing treatment or care, or in their home or workplace. This is especially useful for example where a person is undergoing a course of chemotherapy and the system helps to protect the person's health by killing airborne viruses that the person would otherwise be exposed to. The air sterilisation unit 1 can be used as part of an air conditioning system to sterilise air that is circulated around a building such as a home, shop, restaurant, hospital, workplace, or school. The air flow path 20 may be located only partially within the housing 2 and may pass through other air transfer components such as ducts or pipework.

In FIG. 5 there is a shown a further embodiment of an air sterilisation unit indicated by reference numeral 201. The air sterilisation unit 201 comprises a housing 202, air intake arrangement 203, air outlet arrangement 204, and air outlet chamber 223. The air sterilisation unit 201 further has an arrangement 205 for sterilising air within the housing 202, an air flow generation arrangement 230, and a droplet separator 226.

In this embodiment, the arrangement 205 for sterilising air within the housing 202 is configured to activate hydrogen peroxide by using UV lamps to kill pathogens in the air located within the air sterilisation unit 201. To achieve this, the arrangement 205 for sterilising air comprises four UV lamps 215 located in the air outlet chamber 223. In other embodiments, there may be more or less than four UV lamps. The UV lamps 215 are located between the air flow generation arrangement 230 and the droplet separator 226. The UV lamps 215 reduce hydrogen peroxide via activation and can reduce the level of hydrogen peroxide to less than 1 part per million (PPM).

In this embodiment, the air sterilisation unit 201 has a reservoir 235 of hydrogen peroxide to supply hydrogen peroxide to the air within the air sterilisation unit 201. The hydrogen peroxide in the reservoir 235 is supplied to the air outlet chamber 223. The reservoir is also configured to be manually topped up with hydrogen peroxide when needed. The air sterilisation unit 201 further has a means 236 for supplying the hydrogen peroxide from the hydrogen peroxide reservoir 235 to a space between the air flow generation arrangement 230 and the droplet separator 226. Specifically, the means 236 for supplying the hydrogen peroxide can supply hydrogen peroxide from the hydrogen peroxide reservoir 235 to the air outlet chamber 223. The air outlet chamber 223 is a space that contains UV light from the UV lamps 215. The means 236 for supplying hydrogen peroxide is or comprises a wick (not shown).

In this embodiment, the air sterilisation unit 201 further has a means 237 for preventing UV light from escaping the air sterilisation unit. The means 237 for preventing UV light from escaping the air sterilisation unit 201 prevents at least some, most preferably most, of the UV light from escaping the air sterilisation unit via the air outlet arrangement 204.

The means 237 for preventing UV light from escaping the air sterilisation unit 201 comprises a protective layer 238. The protective layer 238 is located between the UV lamps 215 and the air outlet arrangement 204. The protective layer 238 permits movement of air across the layer but restricts movement of UV light across the layer.

The air sterilisation unit 201 has porous media 239 configured to receive hydrogen peroxide from the hydrogen peroxide reservoir 235. The porous media 239 is located in the air outlet chamber 223, and between the droplet separator 226 and air outlet arrangement 204. The porous media 239 may be formed from ceramic or fibrous material. The porous media 239 can retain a quantity of hydrogen peroxide on or within the media 239. Air moves through the porous media 239 towards the air outlet arrangement 204 and this generates hydrogen peroxide vapour in the air outlet chamber 223.

In FIG. 6, there is shown an embodiment of an air sterilisation unit according to the invention, indicated generally by reference numeral 301. In this embodiment, the air sterilisation unit 301 has two filters 340a, 340b. The filters 340a, 340b are located between the air intake arrangement 303 and the air outlet arrangement 304. More specifically, the filters 340a, 340b are located closer to the air intake arrangement 303 than the air outlet arrangement 304. The filters 340a, 340b are arranged such that air that enters the air intake arrangement 303 contacts the filter 340a before other components of the air sterilisation unit 301, for example, components in the air outlet chamber 323. The air sterilisation unit 301 has a first filter 340a which is a filter for large dust extraction, and a second filter 340b which is a carbon filter. In this illustrated embodiment, there is no packing material. The air sterilisation unit 301 can therefore be smaller in size than the first and second embodiments which include packing material and may even be formed as a desk unit. Advantageously, this unit can be added to an HVAC system as part of a closed-loop system.

In FIGS. 7 and 8, there is shown an embodiment of an air sterilisation unit in accordance with the invention, indicated generally by reference numeral 401. FIG. 7 is a view of the exterior of the air sterilisation unit and FIG. 8 is a cross section displaying the interior of the air sterilisation unit 401.

In this embodiment the air sterilisation unit 401 comprises a housing 402 having an air intake 403, an air outlet 404 and an air purification arrangement 405 for sterilising air within the housing. The arrangement includes a chamber within which air is sterilised by exposure to a liquid, such as water, containing hydrogen peroxide. The liquid is housed in a replaceable cartridge 406 at the base of the air sterilisation unit 401 and accesses the sterilisation chamber via a piezo pump 407. The cartridge 406 further incudes a wick 408 for supplying the hydrogen peroxide water mix from the cartridge to the chamber 405. The concentration of hydrogen peroxide is maintained, preferably at less than 9% within the chamber 405.

Three UVc lamps 409, within the housing 402, are configured to activate the hydrogen peroxide by killing pathogens in the air located within the air sterilisation unit. The three UVc lamps 409 are located in a removable housing for ease of access for replacement of the lamps.

The UVc lamps 409 reduce hydrogen peroxide via activation and can reduce the level of hydrogen peroxide to less than 1 PPM. In operation, therefore, Hydrogen Peroxide is supplied from the cartridge 406 to the chamber 405 that contains the UVc lamps 409. The hydrogen peroxide is in a fluid form but is converted into the vapour by the movement of air through the air sterilisation unit 401. The hydrogen peroxide vapour then reacts with the UV light from the UV lamps 408. This in turn kills/deactivates 99.9% of pathogens and organic compounds in a single air pass. A HEPA filter 410 is also provided.

Air entering the housing 402 at the air intake 403 is drawn through the chamber 405 and exits the housing 402 through the air outlet 404. This air flow is caused by an airflow generation arrangement 411 in the form of a centrifugal fan. The fan is located on the opposite side of the chamber 405 from the air intake 403, towards the top of the air sterilisation unit 401. The controls and the necessary electronics for the fan and the UVc lamps 409 are located in an electrical chamber located at the top of the air sterilisation unit 401. An on/off switch 413 is located adjacent to the electrical chamber 412 on the top surface of the air sterilisation unit.

The air sterilisation unit 401 is portable and can be carried using the strap 414.

Features may be combined from each of the four embodiments of air sterilisation units 1, 201, 301, 401 herein disclosed in any combination. For example, the filters 340a, 340b of the third embodiment air sterilisation unit 301 could be added to the first or second embodiment.

In the preceding discussion of the invention, unless stated to the contrary, the disclosure of alternative values for the upper or lower limit of the permitted range of a parameter, coupled with an indication that one of the values is more highly preferred than the other, is to be construed as an implied statement that each intermediate value of the parameter, lying between the more preferred and the less preferred of the alternatives, is itself preferred to the less preferred value and also to each value lying between the less preferred value and the intermediate value.

The features disclosed in the foregoing description or the following drawings, expressed in their specific forms or in terms of a means for performing a disclosed function, or a method or a process of attaining the disclosed result, as appropriate, may separately, or in any combination of such features be utilised for realising the invention in diverse forms thereof as defined in the appended claims.

1. An air sterilisation unit comprising: a housing having an air intake means arranged to allow contaminated air to enter the housing, and an air outlet means arranged to allow sterilised air to exit the housing; and air sterilisation means arranged to sterilise air within the housing by entrapping or removing undesirable matter from the air flowing from the air intake means to the air outlet means by means of a fluid comprising less than 9% hydrogen peroxide, wherein the air sterilisation unit is a standalone and portable unit, wherein the air sterilisation unit comprises a means for manipulating fluid-particle contact within the housing so as to increase fluid-particle contact within the housing, wherein the air sterilisation unit comprises an air filtration means in the form of an outer air filtration means adapted to filter particles at the point of entry into the air sterilisation unit, and wherein the air sterilisation unit comprises an air flow generation means arranged to enable air to be drawn through the air sterilisation unit, wherein the air flow generation means is located within the housing of the air sterilisation unit.

2. (canceled)

3. The air sterilisation unit as claimed in claim comprising an air sterilisation chamber.

4. The air sterilisation unit as claimed in claim 3, comprising a fluid supply means arranged to supply fluid within the air sterilisation chamber.

5. The air sterilisation unit as claimed in claim 4, wherein the fluid supply means is configurable to supply droplets of fluid to air within the housing.

6. The air sterilisation unit as claimed in claim 4, comprising a fluid delivery means configured to supply fluid from one or more parts of the air sterilisation unit to one or more other parts of the air sterilisation unit including the fluid supply means.

7. The air sterilisation unit as claimed in claim 6, wherein the fluid delivery means comprises a fluid reservoir configured to contain fluid and the fluid delivery means is configurable to deliver droplets of fluid from the fluid reservoir to a means for sterilising air within the housing.

8. The air sterilisation unit as claimed in claim 7, wherein the fluid reservoir comprises a fluid sterilisation means which is configurable to sterilise and/or disinfect fluid within the fluid reservoir.

9. The air sterilisation unit as claimed in claim 8, wherein the fluid sterilisation means comprises one or more UV lamps configurable to sterilise and/or disinfect the fluid.

10. The air sterilisation unit as claimed in claim 8, wherein the fluid sterilisation means comprises a means for supplying a sterilisation agent to the fluid reservoir so as to sterilise and/or disinfect the fluid.

11. The air sterilisation unit as claimed in claim 10, wherein the fluid sterilisation means comprises a means for generating sterilisation agent in the form of hydrogen peroxide and/or singlet oxygen.

12. The air sterilisation unit as claimed in claim 10, wherein the fluid sterilisation means comprises one or more base materials and/or reactants configurable to react to produce singlet oxygen and activation means configurable to activate the one or more base materials.

13. (canceled)

14. The air sterilisation unit as claimed in claim 1, wherein the means for manipulating fluid-particle contact within the housing is configurable to manipulate the speed and/or direction of airflow through the housing so as to reduce the speed of airflow through the housing.

15. (canceled)

16. The air sterilisation unit as claimed in claim 1, wherein the means for manipulating fluid-particle contact within the housing comprises packing material, which is configured to slow airflow through the housing such that fluid-particle contact and/or solvent-particle contact is increased.

17. The air sterilisation unit as claimed in claim 6, wherein; the means for sterilising air within the housing is located in a middle or lower portion of the housing.

18. The air sterilisation unit as claimed in claim 1, comprising a droplet separator, which is adapted to separate any fluid entrained in the air within the housing.

19. The air sterilisation unit, as claimed in claim 6, wherein the fluid supply means is in fluid communication with the fluid reservoir via a fluid flow path, and comprises one or more fluid outlet apertures located at an uppermost portion of the air sterilisation chamber.

20. (canceled)

21. The air sterilisation unit as claimed in claim 1, wherein the air flow generation means is located within or adjacent to the air outlet chamber.

22. The air sterilisation unit as claimed in claim 1, comprising a fluid filtration means configured to remove undesirable matter and/or sediment from fluid travelling into, though, within and/or out of the fluid reservoir.

23. The air sterilisation unit as claimed in claim 1, comprising air filtration means in the form of an intake air filtration means adapted to filter particles such as dust and/or pollen within the air sterilisation unit.

24. An air processing system including a heating, ventilation, conditioning and/or sterilisation system having one or more air sterilisation units as claimed in claim 1.

25. A method for sterilising air, using an air sterilisation unit as claimed in claim 1, the method comprising: generating a flow of air and generating a flow of fluid, within said air sterilisation unit, wherein at least part of the flow of fluid is configured to flow countercurrent to the flow of air.

26. A fluid for use with the air purification unit as claimed in claim 1, in the form of a solvent configurable to absorb and/or entrap undesirable matter suspended in the air within the housing.

27. The fluid as claimed in claim 22 in the form of water comprising 19% hydrogen peroxide.
